SEQUENCE LISTING (2 sequences)

SEQ ID NO: 1 - protein, 9 aa, Artificial
  Streptavidin-binding sequence
AWRHPQFGG

SEQ ID NO: 2 - protein, 8 aa, Artificial
  Streptavidin-binding sequence
WSHPQFEK

(19) United States
(12) Patent Application Publication
(10) Pub. No.: US 2016/0115514 A1
(43) Pub. Date: Apr. 28, 2016

(54) METHOD FOR PREPARATIVE IN VITRO PROTEIN BIOSYNTHESIS

(71) Applicant: Rina-Netzwerk RNA Technology GmbH, Berlin (DE)

(72) Inventors: Jan Strey, Berlin (DE); Helmut Merk, Berlin (DE); Wolfgang Stiege, Berlin (DE)

(21) Appl. No.: 14/728,320

(22) Filed: Jun. 2, 2015

Related U.S. Application Data
(63) Continuation of application No. 14/152,221, filed on Jan. 10, 2014, now abandoned.
Application No. 14/152,221 is a continuation of application No. 13/866,270, filed on Apr. 19, 2013, now abandoned.
Application No. 13/866,270 is a continuation of application No. 13/443,120, filed on Apr. 10, 2012, now abandoned.
Application No. 13/443,120 is a continuation of application No. 12/684,333, filed on Jan. 8, 2010, now abandoned.
Application No. 12/684,333 is a continuation of application No. 11/169,273, filed on Jun. 28, 2005, now abandoned.

(30) Foreign Application Priority Data
Jun. 30, 2004 (DE) 10 2004 032 460.3

(51) Int. Cl. C12P 21/00

(52) U.S. Cl. CPC C12P 21/00

(57) ABSTRACT

The invention relates to a method for preparative in vitro protein synthesis in a cell-free transcription/translation system, comprising the following steps: a) in a reaction vessel, a reaction solution is prepared, comprising the following synthesis substances: components of the transcription/translation apparatus for a defined-protein, amino acids, and metabolic components supplying energy and being necessary for the synthesis of the defined protein, b) the synthesis is performed in the reaction vessel in a defined period of time, c) after expiration of the defined period of time, the reaction solution is subjected to a separation step, in which generated low-molecular metabolic products are separated from the solution (and extracted).

### FIELD OF THE INVENTION

The invention relates to a method for preparative in vitro protein synthesis in a cell-free transcription/translation system, comprising the following steps: a) in a reaction vessel, a reaction solution is prepared, comprising the following synthesis substances: components of the transcription/translation apparatus for a defined protein, amino acids, and metabolic components supplying energy and being necessary for the synthesis of the defined protein, b) the synthesis is performed in the reaction vessel in a defined period of time, c) after expiration of the defined period of time, the reaction solution is subjected to a separation step, in which generated metabolic products are separated from the solution (and extracted). The term protein synthesis means in this invention the expression of the protein.

### PRIOR ART

Methods for the cell-free expression of proteins are for instance known in the art from the documents EP 0312 617 B1, EP 0401 369 B1 and EP 0593 757 B1.

According thereto, the components necessary for [[a]] transcription and/or translation are incubated together with a nucleic acid strand coding for a desired protein in a reaction vessel and after the expression, the polypeptides/proteins are isolated from the reaction solution. The components necessary for the transcription as well as for the translation can easily be extracted from the supernatants of prokaryotic or eukaryotic cell lysates after a 30,000 g (“S-30”) or 10,000 g (“S-10”) centrifugation. The so-called S-30 or S-10 extract contains all the components necessary for transcription and translation, except low-molecular components.

In most cases, the gene or the nucleic acid strand coding for the protein is under the control of a T7 promoter. This has the advantage that by using rifampicin, existing E. coli RNA polymerase can be inhibited, and thus any endogenous E. coli DNA originating from the S30 extract or from the vector preparation is not transcribed. If, however, a gene under the control of an E. coli promoter is expressed, an E. coli polymerase can be used, if not yet present in the extract, which may lead to a co-expression of any endogenous E. coli DNA and thus to undesired endogenous proteins. The expression typically takes place at 37° C.; it may, however, also be made at temperatures from 17° C. to 45° C. The adjustment of the temperature is in particular recommended for the expression of proteins, in which a complex secondary/tertiary structure is to be formed. By lowering the temperature, the synthesis rate can be reduced, and thus the proteins are given the opportunity to correctly fold, in order to obtain a functional/active protein. Further, influence can be obtained on the formation of disulfide bridges within the expressed proteins by the reduction potential of the reaction solution, by the addition of, for instance, dithiothreitol (DTT) and/or oxidized/reduced glutathione.

Before every new protein synthesis, the respective systems should ideally be subjected to an optimization. Thereby, the concentrations of bivalent magnesium ions (Mg2+), of RNA/DNA polymerase and of the coding nucleic acid strand serving as a matrix are varied.

In the method disclosed in the document EP 0312 617 B1 for the cell-free expression of proteins, the nucleic acid strand coding for the protein is added to the reaction solution as mRNA. Thus, for preparing polypeptides in the cell-free system, only the components of the translation apparatus necessary for the translation, in particular ribosomes, initiation, elongation, release factors and aminoacyl-tRNA synthetases as well as amino acids and ATP and GTP as energy-supplying substances need to be brought into a reaction vessel. In the subsequent polypeptide/protein synthesis, in addition to the generation of polypeptides/proteins, low-molecular substances will also be generated, such as ADP, AMP, GDP, GMP and inorganic phosphates under consumption of the energy-supplying substances ATP and GTP and of amino acids. This will lead to a halt in the reaction after the consumption of ATP or GTP or of an amino acid or by the generated low-molecular substances acting as inhibitors. In order to avoid this, the document EP 0312 617 B1 discloses that the substances consumed during the translation are moved out during the translation and simultaneously the energy-supplying substances and the amino acids are introduced for maintaining the initial concentrations.

In contrast thereto, the document EP 0401 369 B1 discloses a method, wherein the nucleic acid strand coding for the protein is added to the reaction solution as mRNA or DNA. The latter has the advantage that DNA is substantially more stable than mRNA, and the necessary transcription process of the DNA into RNA before the reaction is not necessary. Rather, the DNA, e.g. as a vector or a linear construct, can directly be used. By using the DNA, the cell-free expression system must include, in addition to the above translation factors, also the transcription factors necessary for the transcription of the DNA into RNA, such as RNA polymerase, sigma factor or rho protein and the nucleotides ATP, UTP, GTP and CTP. Here, too, the low-molecular substances consumed during the transcription/translation, such as ADP, AMP, GDP, GMP and inorganic phosphates, have to be moved out during translation, and simultaneously the energy-supplying substances, nucleotides and the amino acids have to be introduced for maintaining the initial concentration. From the document EP 0593 757 B1 it is known to separate, beside the consumed low-molecular substances, also the expressed polypeptides from the reaction solution by an ultrafiltration barrier during the translation. Therefore, these methods are continuous synthesis methods.

In the continuous synthesis methods, the obtained long reaction times are per se advantageous with regard to yield, but in turn have disadvantages, too. Firstly, the quality of the newly synthesized proteins are negatively affected by the long retention time, for instance because of degradation, (re-)precipitation, or, when using isotope-marked amino acids, undesired distribution of the isotopes on other amino acid species (caused by amino acid metabolism). On the other hand, for the (continuous) addition of consumed substances, transport gradients over membranes and the like have to be expected, and for this purpose the expensive low-molecular substances, such as energy components, have to be employed in relatively high amounts.

From practice, batch methods for the cell-free protein biosynthesis are known in the art, wherein during the reaction time neither protein products are separated nor consumed substances are added. The initial kinetic conditions are fast, however the time duration is short, so that relatively little protein is obtained. Therefore, these batch methods are only used for analytical and not for preparative purposes.

### SUMMARY OF THE INVENTION

It is the technical object of the invention to provide a method for preparative in vitro protein synthesis, which guarantees high yields with fast kinetics (high productivity) simultaneously with high quality of the expressed proteins and reduced consumption of expensive (energy) components compared to continuous systems.

### BRIEF DESCRIPTION OF THE DRAWINGS

FIG. 1 is a schematic representation of an SDS-PAGE analysis of a protein product prepared according to an embodiment of the invention.

FIG. 2 is a schematic representation of an apparatus according to an embodiment of the invention, comprising a reactor module 1, a recycling module 2, means 3 for moving solutions, a circle line 4, a separation module 5, a switching means 6, and a by-pass 7.

### DETAILED DESCRIPTION OF THE PREFERRED EMBODIMENTS

For achieving this technical object, the invention teaches a method for preparative in vitro protein synthesis of an expression product in a cell-free transcription/translation system, comprising the following steps: a) in a reaction vessel, a reaction solution is prepared, comprising the following synthesis substances: components of the transcription/translation apparatus for a defined expression product, amino acids, and metabolic components supplying energy and being necessary for the synthesis of the defined protein, b) the synthesis is performed in the reaction vessel in a defined period of time, without separating generated substances and without adding consumed synthesis substances within the defined period of time, c) after expiration of the defined period of time, the reaction solution is subjected to a separation step, in which generated low-molecular metabolic products and/or reaction inhibitors are separated from the solution, d) immediately before, after or at the same time as step c), consumed synthesis substances are supplemented, e) steps b), c) and d) are repeated at least once with the reaction solution of step d), and at the last execution of step b), steps c) and d) may be left out.

Expression products are mainly proteins. Reaction inhibitors are substances, which are contained in the reaction solution and/or which are generated during the synthesis and reduce the reaction speed (kinetics of the synthesis) or completely prevent the synthesis, compared to a reaction solution without the reaction inhibitors. The term reaction inhibitors in the meaning of the invention also comprises however components undesired for other reasons.

In principle, the solution obtained with the last execution of step c) is already suitable for various purposes, for instance analytical purposes. If, however, the expression product is needed in a purified form, it may be separated from the solution in step c) or subsequently to step e). This may take place for instance by using a mobile or immobilized matrix. The mode of functioning of such a matrix may be based on any purification methods known for the binding of expression products, such as ion exchange, affinity, antigen/antibody interaction, and hydrophobic/hydrophilic interaction. Therein, the suitable molecules are bound to the surface of a substrate. For a particularly efficient separation, they may be co-expressed with one or several markers, e.g. in the form of several N or C-terminal successive histidines, or one or several other proteins, e.g. glutathione, as a so-called fusion protein. The matrix then includes a binding partner specific for this marker/protein, which permits an efficient binding of the chimeric fusion protein by the marker/fusion partner to the matrix. The matrix may contain anion or cation exchange material or hydroxyapatite. If the proteins are expressed as fusion proteins, and the fusion partners are placed N, C-terminally or within the expressed protein, a matrix may be used, which specifically binds the fusion partner. The protein may contain N or C-terminally several successive histidines, in particular 3 to 12, preferably 5 to 9, most preferably 6, and the matrix may then carry a metal-chelate compound, in particular with bivalent metal ions, preferably copper and/or nickel ions. The protein may contain N or C-terminally glutathione-S-transferase as a fusion partner, and glutathione may be coupled to the matrix. The protein may contain an amino acid sequence permitting a binding to streptavidin, preferably the amino acid sequence AWRHPQFGG (SEQ ID No.: 1), most preferably the amino acid sequence WSHPQFEK (SEQ ID No.: 2), and then streptavidin may be coupled to the matrix.

In principle, the components to be used are known from the prior art. The translation apparatus comprises, in particular, ribosomes, initiation, elongation, release factors and aminoacyl-tRNA synthetases. Therewith (and with further components), the translation of mRNA coding for a protein to be synthesized can be performed. When using DNA coding for the protein to be synthesized, transcription factors for the transcription of the DNA into RNA are necessary, for example, RNA polymerase, sigma factor or rho protein and the nucleotides ATP, UTP, GTP and CTP. The necessary metabolic components of the reaction are selected from the not closed group “ATP, UTP, GTP and CTP, pyrophosphate, amino acids and mixtures of these substances”. The used amino acids may be natural amino acids, but also chemically derivatized non-natural amino acids or isotope-marked amino acids. Low-molecular metabolic products, which are partially or completely (related to a metabolic product species as well as to the totality of the metabolic products) separated or reduced in the recycling step c), are for instance ADP, AMP, GDP, GMP and inorganic phosphate. Low-molecular metabolic products have a molecular weight of less than 10,000 Da, preferably less than 8,000 Da, and most preferably less than 5,000 Da. They may have a molecular weight above 2,000 Da.

The addition of consumed synthesis substances before the separation step can be made in cases where the synthesis substances are high-molecular synthesis substances. They have molecular weights exceeding the molecular weights of the low-molecular metabolic products described above.

The method according to the invention can in principle be executed with prokaryotic as well as with eukaryotic systems. The components necessary for the ranscription/translation can easily be extracted from the supernatants of prokaryotic or eukaryotic cell lysates after a 30,000 g (“S-30”) or 10,000 g (“S-10”) centrifugation. This so-called S-30 or S-10 extract contains all components being essential for the transcription and translation.

Steps b), c) and d) can be repeated one to ten times, preferably one to five times. The defined period of time may be between 0.1 and 10 hours, preferably between 0.5 and 3 hours. Step c) may be executed by means of gel filtration, ultrafiltration, dialysis, diafiltration or matrices having selective binding properties for low-molecular metabolic products and/or reaction inhibitors. The methods gel filtration, ultrafiltration, dialysis and diafiltration are well known to one skilled in the art. For instance, for separating phosphate, Sevelamer® HCl or Renagel® may be used as matrices. Reaction inhibitors may be matrices selectively binding these reaction inhibitors, and the above explanations regarding the separation of expression products apply in an analogous manner.

Basically, the method according to the invention is a repetitive batch method, wherein a batch is repeated with the same reaction solution after an interposed recycling step, in which low-molecular metabolic products are separated from the reaction solution, and consumed substances are added. By means of the invention, on the one hand, shorter reaction times than those of continuous methods are obtained. This results in an improved quality of the product protein. Further, comparatively less low-molecular substances, in particular energy suppliers, have to be used, since concentration gradients are not necessary. Only a supplementation, i.e. an addition is achieved until a defined initial concentration, is required. Nevertheless, high yields with fast kinetics and consequently high productivities are obtained.

Another embodiment of the invention having an independent importance relates to a method for preparative in vitro protein synthesis in a cell-free transcription/translation system, comprising the following steps: A) in a reaction vessel, a reaction solution is prepared, comprising the following synthesis substances: components of the transcription/translation apparatus for a defined first expression product, optionally components of the transcription/translation apparatus for a defined second expression product being different from the first expression product, amino acids, and metabolic components supplying energy and being necessary for the synthesis, B) the synthesis of the first protein is performed in the reaction vessel in a first defined period of time, without adding consumed synthesis substances within the first defined period of time, and C) optionally generated low-molecular metabolic products are separated from the solution, D) after expiration of the defined period of time, the reaction solution is supplemented with consumed synthesis substances and, as far as not added already in step a), reacted with components of the transcription/translation apparatus for the defined second expression product, E) the synthesis of the second protein is performed in the reaction vessel in a second defined period of time, without separating generated substances and without adding consumed synthesis substances within the defined period of time.

Subsequently, the expression product may be separated from the solution, the solution containing the expression product may, however, also immediately be used for other purposes, for instance, analytics or for screening of a substance library without such a separation. In principle, however, the method of claims 1 to 5 may follow, beginning with step c) thereof. Step e) may be omitted. The explanations given above for the method according to one of claims 1 to 5 apply in an analogous manner.

In this embodiment of the invention, various “programmings” are possible. “Programming” refers to the way in which the synthesis of the various expression products in the various steps is controlled.

The transcription/translation apparati for the first expression product and the second expression product may include various first and second regulatory sequences, and a first gene sequence coding for the first expression product is under control the of the first regulatory sequence and a second gene sequence coding for the second expression product is under the control of the second regulatory sequence.

In the embodiment comprising components of the transcription/translation apparatus for a defined second expression product that is different from the first expression product in step A), the second regulatory sequence can be inhibited in step B), and the first regulatory sequence can be inhibited in step E). In the embodiment comprising the addition of the components of the transcription/translation apparatus for the defined second expression product in step D), the first regulatory sequence can be inhibited in step E).

In this embodiment of the invention, too, a repetitive batch method is used, but various expression products, for instance proteins, are obtained in various steps. The second expression product typically is the actually desired product protein. The first expression product, however, is an auxiliary substance, such as translation factors, folding helper proteins, interaction partners, or tRNA. Such substances are helpful for the generation of the product protein, for instance with regard to yield, solubility or functionality. First expression products may, for instance, be chaperones promoting the solubility of the protein product. Insofar, the term synthesis also comprises the term maturation of a protein.

In principle, the solution may also be concentrated in step c) of claim 5 or step C) of claim 6, for instance by dialysis against a PEG solution.

In the following the invention will be explained in more detail, based on examples representing embodiments only.

### Example 1

### Simple Repetition with One Recycling Step

0.5 ml of a reaction solution for the cell-free protein biosynthesis, containing 175 μl S-mix, 150 μl T-mix, 40 μl E-mix (available as components of the “RiNA in-vitro-PBS Kit”, Cat. No. P-1102-14, RiNA GmbH, Berlin, Germany), 63 μM ¹⁴C leucine (100 dpm/pmol), 5 nM plasmid DNA, coding for the elongation factor Ts from E. coli, and RNase free water ad 0.5 ml, were reduced to 50% (250 μl) after incubation (1.5 h, 37° C.) by means of ultrafiltration (10 kDa membrane), and thereafter reacted with 250 μl supplementation mix of the following composition: 100 mM HEPES (pH 7.6), 200 mM potassium acetate, 100 mM ammonium acetate, 46 mM magnesium chloride, 0.2 mM EDTA, 0.04% sodium azide (w/v), 10 mM DTT, 20 04 GDP, 8% PEG3000 (w/v), 200 μM folic acid, 1.2 mM each of all 20 amino acids, 126 μM ¹⁴C leucine, 2 mM each of ATP and GTP, 1 mM each of UTP and CTP, 60 mM phosphoenolpyruvate and 20 mM acetyl phosphate. The following second synthesis took place for 1.5 h at 37° C. The obtained amounts of EF-Ts are (in total) 114 μg after the first synthesis and 221 μg after the second synthesis. The quantification was performed by determination of the integration of applied radioactively marked ¹⁴C leucine.

### Example 2

### Quadruple Repetition of a Batch Reaction with Four Recycling Steps

1 ml of a reaction solution for the cell-free protein biosynthesis, containing 350 μl S-mix, 80 μl E-mix (available as components of the “RiNA in-vitro-PBS Kit”, Cat. No. P-1102-14, RiNA GmbH, Berlin, Germany), 35 mM HEPES (pH 7.6), 70 mM potassium acetate, 35 mM ammonium acetate, 10 mM magnesium chloride, 0.07 mM EDTA, 0.014% sodium azide (w/v), 5 mM DTT, 100 μM folic acid, 1.2 mM each of all 20 amino acids, 63 μM ¹⁴C leucine, 5 nM plasmid DNA, coding for the elongation factor Ts from E. coli, and RNase free water ad 1 ml, were gel-filtrated after incubation (1.5 h, 37° C.) by a Sephadex matrix (G-25), reduced to 50% of the original volume (500 μl) by means of ultrafiltration (10 kDa membrane), and thereafter reacted with 500 μl supplementation mix of the following composition: 100 mM HEPES (pH 7.6), 200 mM potassium acetate, 100 mM ammonium acetate, 26 mM magnesium chloride, 0.2 mM EDTA, 0.04% sodium azide (w/v), 10 mM DTT, 20 μM GDP, 200 μM folic acid, 2.4 mM each of all 20 amino acids, 126 μM ¹⁴C leucine, 2 mM each of ATP and GTP, 1 mM each of UTP and CTP, 60 mM phosphoenolpyruvate and 20 mM acetyl phosphate. The following second synthesis took place for 1.5 h at 37° C. The recycling step (gel filtration, ultrafiltration, supplementation) and the synthesis step were then repeated several times (recycling: another three times=four times in total; synthesis: another three times=five times in total). The obtained amounts of EF-Ts are (in total) 171 μg after the first synthesis, 315 μg after the second synthesis, 447 μg after the third synthesis, 561 μg after the fourth synthesis, and 650 μg after the fifth synthesis. The quantification was performed by determination of the integration of applied radioactively marked ¹⁴C leucine.

### Example 3

### Double Repetition of a High-Yield Batch Reaction with Two Recycling Steps

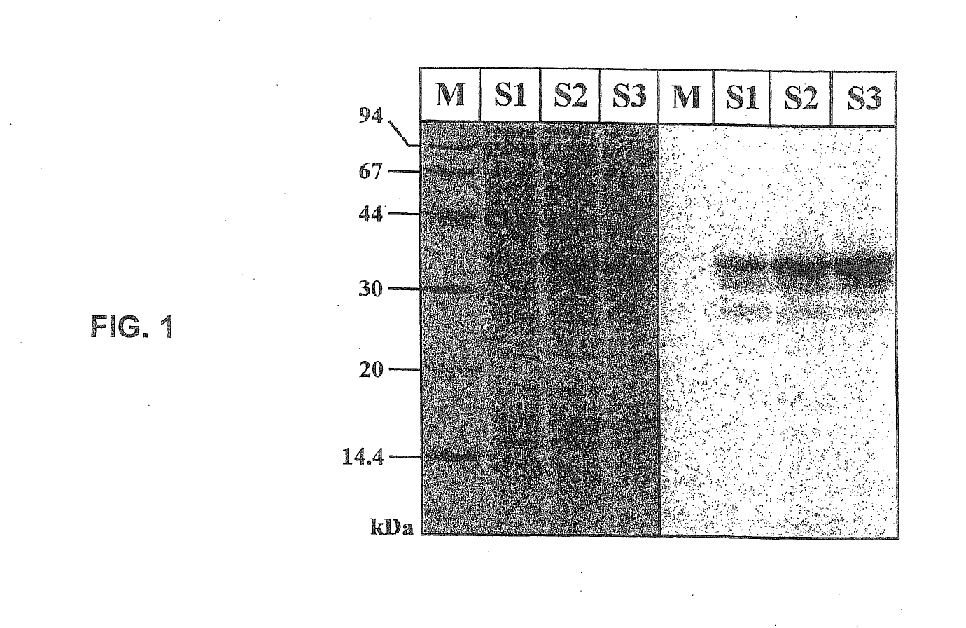

1.8 ml of a reaction solution for the cell-free protein biosynthesis were prepared as follows: 720 μl EasyXPress® Reaction Buffer were reacted with 630 μl E. coli extract (both components included in the EasyXPress® Protein Synthesis Maxi Kit, Cat. No. 32506, Qiagen GmbH, Hilden, Germany), 63 μM ¹⁴C leucine (100 dpm/pmol), 10 mM plasmid DNA, coding for the elongation factor Ts from E. coli, and RNase free water ad 1.8 ml. The reaction was then concentrated up by means of ultrafiltration (10 kDa membrane) to 1 ml, and incubated for 1 h at 37° C. After this first synthesis phase, the batch was gel-filtrated over a Nap-10 column (Sephadex G-25) and supplemented with 300 μl of a solution containing 300 mM HEPES (pH 7.6), 600 mM potassium acetate, 300 mM ammonium acetate, 114 mM magnesium chloride, 0.6 mM EDTA, 0.12% sodium azide (w/v), 6 mM DTT, 60 μM GDP, 24% PEG3000 (w/v), 600 μM folic acid, 7.2 mM each of all 20 amino acids, 380 μM ¹⁴C leucine, 10.2 mM each of ATP and GTP, 5.1 mM each of UTP and CTP, 306 mM phosphoenolpyruvate and 102 mM acetyl phosphate. The following second synthesis took place for 1.0 h at 37° C. Thereafter the recycling step (gel filtration, supplementation) and the synthesis step were repeated once again, and the supplementation mix now had the following composition: 300 mM HEPES (pH 7.6), 600 mM potassium acetate, 300 mM ammonium acetate, 78 mM magnesium chloride, 0.6 mM EDTA, 0.12% sodium azide (w/v), 6 mM DTT, 60 μM GDP, 24% PEG3000 (w/v), 600 μM folic acid, 7.2 mM each of all 20 amino acids, 380 μM ¹⁴C leucine, 6 mM each of ATP and GTP, 3 mM each of UTP and CTP, 180 mM phosphoenolpyruvate and 60 mM acetyl phosphate. In total, three synthesis steps and two recycling steps were passed. The obtained amounts of EF-Ts are (in total) 563 μg after the first synthesis, 1,804 μg after the second synthesis and 2,487 μg after the third synthesis. By repeating twice, therefore, a yield of 4.4 times the first synthesis step was obtained. The quantification was performed by determination of the integration of applied radioactively marked ¹⁴C leucine. FIG. 1 shows an SDS-PAGE analysis of the protein product from this example. On the left-hand side, the Coomassie staining can be seen, and on the right-hand side the autoradiogram is shown. The track M is the molecular weight standard, the tracks S1 to S3 are the three synthesis steps. The theoretical value of the EF-Ts is 31.6 kDA.

### Example 4

### Programming/Conditioning of a Transation System

In a first synthesis step, a gene for the synthesis or quality of the product protein to be generated in the second synthesis step, for instance a gene for a chaperone (promoting solubility for the product protein) is used. The chaperone gene is under control of the E. coli promoter. After completion of the first synthesis step, a recycling step is performed, wherein there is no separation of the expression product (chaperone), but only a supplementation and the addition of a gene under control of the T7 promoter for the product protein. Further, an inhibitor of the E. coli RNA polymerase, for instance rifampicin, is added. In the second synthesis step, therefore, there takes place practically exclusively the expression of the product protein, and the latter is obtained with an appreciably improved solubility, because of the presence of the chaperones from the first synthesis step. Alternatively to the inhibition of an RNA polymerase used in the first synthesis step, the concentration of the gene or template used in this step can be reduced for the second synthesis step, for instance by separation or by dilution.

### Example 5

### Apparatus for a Method According to the Invention

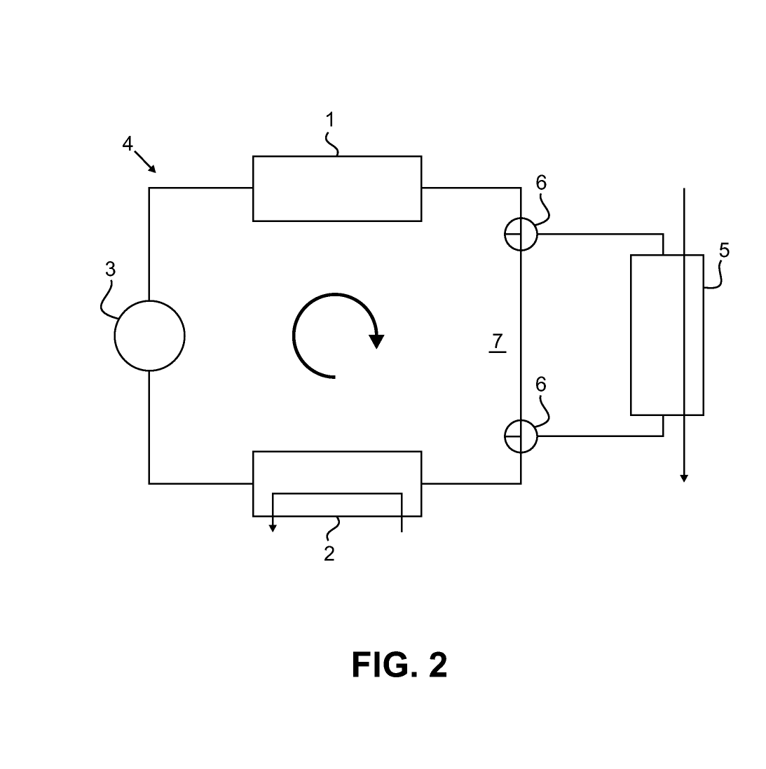

FIG. 2 shows an apparatus being suitable for the invention. There is shown a reaction module 1, a recycling module 2, means 3 for moving solutions and a circle line 4. In the reaction module 1, steps b), b′) and/or e′) are performed. In the recycling module 2 follow steps c), d), c′) and/or d′). The means 3 for moving solutions are controlled such that the steps according to the invention take successively place after the defined periods of time. Further, there is a separation module 5, where the expression product can be separated from the solution. There are also provided switching means 6 connecting the separation module 5 into the circle line 4 at the place of the by-pass 7.

1. A method for preparative in vitro protein synthesis of an expression product in a cell-free transcription/translation system, comprising the following steps:
a) in a reaction vessel, a reaction solution is prepared, comprising the following synthesis substances: components of the transcription/translation apparatus for a defined expression product, amino acids, and metabolic components supplying energy and being necessary for the synthesis of the defined protein,
b) the synthesis is performed in the reaction vessel in a defined period of time, without separating generated substances and without adding consumed synthesis substances within the defined period of time,
c) after expiration of the defined period of time, the reaction solution is subjected to a separation step, in which generated low-molecular weight metabolic products and/or reaction inhibitors are separated from the solution,
d) immediately before, after or at the same time as step c), consumed synthesis substances are supplemented,
e) steps b), c) and d) are repeated at least once with the reaction solution of step d), wherein at the last execution of step b), the steps c) and d) may be omitted.

2. The method according to claim 1, wherein in step c) and/or subsequently to step e), expression products are separated from the solution.

3. The method according to claim 1, wherein steps b), c) and d) are repeated one to ten times.

4. The method according to claim 1, wherein the defined period of time is between 0.1 and 10 hours, preferably between 0.5 to 3 hours.

5. The method according to claim 1, wherein step c) is executed by means of gel filtration, ultrafiltration, dialysis, diafiltration or matrices having selective binding properties for low-molecular weight metabolic products and/or reaction inhibitors.

6. A method for preparative in vitro protein synthesis in a cell-free transcription/translation system, comprising the following steps:
A) in a reaction vessel, a reaction solution is prepared, comprising the following synthesis substances: components of the transcription/translation apparatus for a defined first expression product, amino acids, and metabolic components supplying energy and being necessary for the synthesis,
B) the synthesis of the first protein is performed in the reaction vessel in a first defined period of time, without adding consumed synthesis substances within the first defined period of time,
C) any generated low-molecular weight metabolic products are separated from the solution,
D) after expiration of the defined period of time, the reaction solution is supplemented with consumed synthesis substances andreacted with components of the transcription/translation apparatus for a defined second expression product,
E) the synthesis of the second protein is performed in the reaction vessel in a second defined period of time, without separating generated substances and without adding consumed synthesis substances within the defined period of time.

7. The method according to claim 6, wherein subsequently to step E) the method according to claim 1 is performed, beginning with step c).

8. The method according to claim 6, wherein the transcription/translation apparatuses for the first expression product and the second expression product include different first and second regulatory sequences, wherein a first gene sequence coding for the first expression product is under the control of the first regulatory sequence and a second gene sequence coding for the second expression product is under the control of the second regulatory sequence.

9. The method according to claim 6 in the embodiment comprising components of transcription/translation apparatus for a defined second expression product being different from the first expression product in step A), wherein the second regulatory sequence is inhibited in step B), and wherein the first regulatory sequence is inhibited in step E).

10. The method according to claim 6 in the embodiment comprising the addition of the components of the transcription/translation apparatus for the defined second expression product in step D), wherein the first regulatory sequence is inhibited in step E).

11. The method according to claim 1, wherein steps b), c) and d) are repeated one to five times.

12. The method according to claim 1, wherein the defined period of time is between 0.5 to 3 hours.

13. The method of claim 6, wherein the components of the transcription/translation apparatus for a defined second expression product are different than for the first expression product.

14. A method for preparative in vitro protein synthesis in a cell-free transcription/translation system, comprising the following steps:
A) in a reaction vessel, a reaction solution is prepared, comprising the following synthesis substances: components of the transcription/translation apparatus for a defined first expression product, components of the transcription/translation apparatus for a defined second expression product being different from the first expression product, amino acids, and metabolic components supplying energy and being necessary for the synthesis, wherein expression of the second expression product is inhibited,
B) the synthesis of the first protein is performed in the reaction vessel in a first defined period of time, without adding consumed synthesis substances within the first defined period of time,
C) any generated low-molecular weight metabolic products are separated from the solution,
D) after expiration of the defined period of time, the reaction solution is supplemented with consumed synthesis substances and reacted with the components of the transcription/translation apparatus for the defined second expression product,
E) the synthesis of the second protein is performed in the reaction vessel in a second defined period of time, without separating generated substances and without adding consumed synthesis substances within the defined period of time.
